(19) United States
(12) Patent Application Publication
(10) Pub. No.: US 2021/0186950 A1
(43) Pub. Date: Jun. 24, 2021

(54) COMBINATIONS COMPRISING TROPIFEXOR AND CENICRIVIROC

(71) Applicant: NOVARTIS AG, Basel (CH)

(72) Inventors: Jessie Gu, Framingham, MA (US); Marcos Pedrosa, Basel (CH); Rowan Stringer, Oberwil BL (CH)

(21) Appl. No.: 17/055,468

(22) PCT Filed: May 28, 2019
(86) PCT No.: PCT/IB2019/054398

§ 371 Date: Nov. 13, 2020

Related U.S. Application Data
(60) Provisional application No. 62/678,448, filed on May 31, 2018.

(51) Int. Cl. A61K 31/46; A61K 31/4178

(52) U.S. Cl. CPC A61K 31/46; A61K 31/4178

(57) ABSTRACT

The invention provides a pharmaceutical combination comprising 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a stereoisomer, enantiomer, pharmaceutically acceptable salt, prodrug, ester thereof or amino acid conjugate thereof; and cenicriviroc or a pharmaceutically acceptable salt, solvate, prodrug or ester thereof; for use in treating or preventing a liver disease or disorder.

### FIELD OF THE INVENTION

The present invention relates to a pharmaceutical combination comprising the farnesoid X receptors (FXRs) agonist tropifexor and cenicriviroc, optionally in the presence of a pharmaceutically acceptable carrier and pharmaceutical compositions comprising them. Furthermore, the invention is directed to the use of such pharmaceutical combinations for treating or preventing fibrotic or cirrhotic diseases or disorders, e.g. liver diseases or disorders, as well as compositions, methods, uses and regimens involving such combinations.

### BACKGROUND OF THE INVENTION

Nonalcoholic fatty liver disease (NAFLD) is the most common cause of chronic liver disease in the Western world (Ratziu et al 2010). The main stages of NAFLD are 1-simple fatty liver (steatosis); 2-non-alcoholic steatohepatitis (NASH), a more serious form of NAFLD; 3-fibrosis, where there is a persistent inflammation in the liver resulting in the generation of fibrous scar tissue around the liver cells and blood vessels; and 4-cirrhosis; this damage is permanent and can lead to liver failure and liver cancer.

NASH includes fat accumulation in the liver, as well as inflammation, which over time can lead to increasing fibrosis, cirrhosis and end stage liver disease. Liver transplantation is the only treatment for advanced cirrhosis with liver failure, and transplantation is increasingly performed in persons suffering from NASH.

Estimates of the worldwide prevalence of NAFLD range from 6.3% to 33% with a median of 20% in the general population. The estimated prevalence of NASH is lower, ranging from 3 to 5% (Younossi et al., Hepatology, Vol. 64, No. 1, 2016). NASH is a worldwide problem with growing prevalence over the last few decades. Over the last decade NASH has risen from uncommon to the second indication for liver transplantation in the US. It is expected to be the leading cause of transplant by 2020 (Wong, et al, Gastro 2015). NASH is highly associated with the metabolic syndrome and Type 2 diabetes mellitus. NASH is a cause of progressive fibrosis and of cirrhosis. Cirrhosis due to NASH increases the risk of hepatocellular carcinoma and hepatocellular cancer. Furthermore, cardiovascular mortality is an important cause of death in NASH patients.

Development of NASH, involves several mechanisms: accumulation of fat in the liver (steatosis), inflammation of the liver, hepatocyte ballooning, and fibrosis. The NAFLD Activity Score (NAS) was developed as a tool to measure changes in NAFLD during therapeutic trials. The score is calculated as the unweighted sum of the scores for steatosis (0-3), lobular inflammation (0-3), and ballooning (0-2).

For preventing or treating such diseases or disorders, a medicament would be particularly efficient if it has an impact on each of these different aspects.

C-C chemokine receptor type 2 (CCR2) and CCR5 play a role in entry of viruses such as Human Immunodeficiency Virus (HIV) into the cell, but also are important for the recruitment of immune cells to sites of injury. Inhibition of this receptor's activity may have an anti-inflammatory effect. And recent data indicate that these receptors may also play a role in promoting hepatic fibrosis. Cenicriviroc (also known as CVC) is (S,E)-8-(4-(2-butoxyethoxy)phenyl)-1-(2-methylpropyl)-N-(4-(((1-propyl-1H-imidazol-5-yl) methyl)sulfinyl)phenyl)-1, 2,3,4-tetrahydrobenzo[b]azocine-5-carboxamide. Cenicriviroc binds to and inhibits the activity of the C-C chemokine receptor type 2 (CCR2) and C-C chemokine receptor type 5 (CCR5) receptors.

In a clinical trial aimed at evaluating the efficacy and safety of cenicriviroc for the treatment of NASH in adults with liver fibrosis, the treatment with CVC shown a reduction of fibrosis but no significant impact on the improvement of NAS. Furthermore CVC can induce fatigue or diarrhea in a small part of the patients (see “Tobira Therapeutics Announces Clinically and Statistically Significant Improvement in Liver Fibrosis from Phase 2b CENTAUR NASH Trial at One Year” Jul. 25, 2016).

When tested in nonalcoholic steatohepatitis patients, obeticholic acid (OCA), a bile-acid mimetic, showed efficacy, in particular a significant improvement in NAS, i.e. strong impact on steatosis with additional effects on inflammation and ballooning. But OCA long term administration raises safety concerns because it can be associated with pruritus, as well as with increased LDL cholesterol (see “Intercept Announces New FLINT Trial Data Showing OCA Treatment Increases Fibrosis Resolution and Cirrhosis Prevention in High-Risk NASH Patients”, Apr. 23, 2015). To avoid the risk of adverse cardiovascular events, concomitant administration of statins may be required for long-term treatment of NASH patients.

The FXR agonist tropifexor is 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid (see Tully et all 2017) and is currently tested in nonalcoholic steatohepatitis patients with fibrosis (see NCT02855164 study). The compound was disclosed for the first time in WO 2012/087519 (Example 1, compound 1-IB of the table on page 125) and it is also known under the name LJN452 and under its International Nonproprietary Name (INN) “Tropifexor”.

Currently there is no approved therapy for NASH.

Therefore, there is a need to provide treatments for fibrotic/cirrhotic diseases or disorders, e.g. liver diseases or disorders, which can address the different aspects of these complex conditions, while demonstrating an acceptable safety and/or tolerability profile. The combination of two or more molecules with different Mechanisms of Action (MoA) might provide additional benefits for improving treatment efficacy and response rates, for preventing or slowing down the progression of NAFLD and NASH.

### SUMMARY OF THE INVENTION

Tropifexor is a highly potent FXR receptor agonist, while cenicriviroc (CVC) is a potent and selective inhibitor of CCR2/5. A combination of tropifexor and CVC has the potential to address metabolic, anti-inflammatory and antifibrotic pathways involved in NASH. Such combinations are described in WO/2018/051230, the disclosure of which is hereby incorporated herein by reference in its entirety for all purposes.

The invention provides new pharmaceutical combinations, containing, separate or together, the FXR agonist 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid , in free form or a pharmaceutically acceptable salt, solvate, prodrug, ester and/or an amino acid conjugate thereof, also known under its INN tropifexor, and cenicriviroc (as herein defined, e.g. in free form or as a pharmaceutically acceptable salt thereof, e.g. cenicriviroc mesylate), for simultaneous, sequentially or separate administration. The pharmaceutical combinations comprise: (i) an amount of 120 μg to about 250 μg, of about 140 μg to about 200 μg of tropifexor, and (ii) an amount of about 150 mg of cenicriviroc. The pharmaceutical combinations comprise: (i) an amount of about 140 μg of tropifexor, and (ii) an amount of about 150 mg of cenicriviroc. There is also provided a medicament, comprising such combinations.

In some aspects, cenicriviroc is cenicriviroc mesylate.

There is also provided pharmaceutical combinations containing, separately or together, (i) tropifexor, and (ii) cenicriviroc (as herein defined, e.g. in free form or as a pharmaceutically acceptable salt thereof, e.g. cenicriviroc mesylate), for simultaneous, sequential or separate administration. The pharmaceutical combinations comprise: (i) an amount of 120 μg to about 250 μg, of about 140 μg to about 200 μg of tropifexor, and (ii) an amount of about 150 mg of cenicriviroc.

Components (i) and (ii) can be administered together, one after the other or separately in one combined unit dose form or in two separate unit dose forms. The unit dose form may also be a fixed combination. Component (i) is to be administered at a dose (e.g. daily dose) of about 120 μg to about 250 μg, of about 140 μg to about 200 μg and component (ii) is to be administered at a dose (e.g. daily dose) of about 150 mg, e.g. at a dose of 150 mg. Component (i) is to be administered at a dose (e.g. daily dose) of 140 μg and component (ii) is to be administered at a dose (e.g. daily dose) of 150 mg.

In some aspects, the pharmaceutical combination is a fixed combination, e.g. a fixed combination comprising (i) tropifexor, and (ii) cenicriviroc (as herein defined, e.g. in free form or as a pharmaceutically acceptable salt thereof, e.g. cenicriviroc mesylate).

In some aspects, Components (i) and (ii), the pharmaceutical combinations as defined herein, are provided for the treatment of a fibrotic disease or disorder, e.g. a liver disease or disorder, e.g. a chronic liver disease or disorder, e.g. a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatis or PBC. Component (i) is to be administered at a dose (e.g. daily dose) of about 120 μg to about 250 μg, of about 140 μg to about 200 μg and component (ii) is to be administered at a dose (e.g. daily dose) of about 150 mg, e.g. at a dose of 150 mg. Component (i) is to be administered at a dose (e.g. daily dose) of 140 μg and component (ii) is to be administered at a dose (e.g. daily dose) of 150 mg.

In other aspects of the invention, Components (i) and (ii), the pharmaceutical combinations as defined herein are provided for slowing, arresting, or reducing the development of a cirrhotic disease or disorder, e.g. a chronic liver disease or disorder, e.g. NAFLD, NASH, liver fibrosis and PBC. Component (i) is to be administered at a dose (e.g. daily dose) of about 120 μg to about 250 μg, about 140 μg to about 200 μg and component (ii) is to be administered at a dose (e.g. daily dose) of about 150 mg, e.g. at a dose of 150 mg. Component (i) is to be administered at a dose (e.g. daily dose) of 140 μg and component (ii) is to be administered at a dose (e.g. daily dose) of 150 mg.

In yet another aspect, Components (i) and (ii), the pharmaceutical combinations as defined herein are provided for preventing or delaying progression of a chronic liver disease or disorder to a more advanced stage or a more serious condition thereof, e.g. for preventing or delaying progression of a chronic liver disease or disorder selected from the group consisting of NAFLD, NASH, hepatic fibrosis and PBC. Component (i) is to be administered at a dose (e.g. daily dose) of about 120 μg to about 250 μg, of about 140 μg to about 200 μg and component (ii) is to be administered at a dose (e.g. daily dose) of about 150 mg, e.g. at a dose of 150 mg. Component (i) is to be administered at a dose (e.g. daily dose) of 140 μg and component (ii) is to be administered at a dose (e.g. daily dose) of 150 mg.

The invention is also directed to pharmaceutical combinations comprising, (i) tropifexor, and (ii) cenicriviroc (as herein above defined, e.g. in free form or as a pharmaceutically acceptable salt or solvate thereof), optionally in the presence of a pharmaceutically acceptable carrier. In some embodiments of the invention, such a pharmaceutical combination is combined unit dose form.

In some aspects, there is provided pharmaceutical combinations comprising (i) tropifexor, and (ii) cenicriviroc (as herein above defined, e.g. in free form or as a pharmaceutically acceptable salt or solvate thereof), in a quantity which is jointly therapeutically effective for use in the treatment or prevention of fibrotic or cirrhotic diseases or disorders, e.g. liver diseases or disorders, e.g. NAFLD, NASH, liver fibrosis or PBC. The pharmaceutical combinations for use as herein defined, comprise: (i) an amount of 120 μg to about 250 μg, of about 140 μg to about 200 μg of tropifexor, and (ii) an amount of about 150 mg of cenicriviroc. The pharmaceutical combinations comprise: (i) an amount of about 140 μg of tropifexor, and (ii) an amount of about 150 mg of cenicriviroc.

Furthermore, the invention relates to such pharmaceutical combinations, e.g. fixed or free combinations, e.g. combined unit doses, for use in treating, preventing or ameliorating a fibrotic or cirrhotic disease or disorder, e.g. a liver disease or disorder

There is provided the use of tropifexor, in combination, e.g. fixed or free combination, with cenicriviroc (or a pharmaceutically acceptable salt, solvate, prodrug and/or ester thereof, e.g. cenicriviroc mesylate), for the manufacture of a medicament for the prevention or treatment of a liver disease or disorder, e.g. a liver disease or disorder selected from the group consisting of NAFLD, NASH, hepatosteatosis, liver fibrosis, cirrhosis, PBC.

There is also provided pharmaceutical combinations for use preventing, delaying or treating a liver disease or disorder, wherein the combination comprises (i) tropifexor and (ii) cenicriviroc (in free form or as a pharmaceutically acceptable salt, solvate, prodrug and/or ester thereof, e.g. cenicriviroc mesylate).

In some aspects of the invention, there is provided pharmaceutical combinations for use in preventing, delaying or treating a chronic liver disease or disorder, e.g. selected from the group consisting of steatosis, NASH, fibrosis and cirrhosis, e.g. steatosis, NASH and/or fibrosis, wherein the combination comprises (i) tropifexor, and (ii) cenicriviroc (in free form or as a pharmaceutically acceptable salt, solvate, prodrug and/or ester thereof, e.g. in free form or as a pharmaceutically acceptable salt thereof, e.g. cenicriviroc mesylate).

There is further provided pharmaceutical combinations comprising (i) tropifexor, and (ii) cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use preventing, delaying or treating NASH.

Furthermore, there is also provided pharmaceutical combinations comprising (i) tropifexor, and (ii) cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use preventing, delaying or treating liver fibrosis.

There is also provided pharmaceutical combinations comprising (i) tropifexor, and (ii) cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use in preventing, delaying or treating hepatosteatosis.

There is further provided pharmaceutical combinations comprising (i) tropifexor and (ii) cenicriviroc (in free form or as a pharmaceutically acceptable salt, solvate, prodrug, and/or ester thereof, e.g. in free form or as a pharmaceutically acceptable salt thereof e.g. cenicriviroc mesylate), for use in preventing, delaying or treating hepatocellular ballooning.

There is also provided pharmaceutical combinations comprising (i) tropifexor, and (ii) cenicriviroc (in free form or as a pharmaceutically acceptable salt, solvate, prodrug, and/or ester thereof, e.g. in free form or as a pharmaceutically acceptable salt thereof, e.g. cenicriviroc mesylate), for use in preventing, delaying or treating PBC.

A further aspect of the present invention is a method for the treatment, delaying or prevention of a fibrotic disease or disorder, e.g. a liver disease or disorder, e.g. chronic liver disease or disorder, comprising administering a therapeutically effective amount of combination of (i) tropifexor, and (ii) cenicriviroc (in free form or as a pharmaceutically acceptable salt, solvate, prodrug, and/or ester thereof, e.g. in free form or as a pharmaceutically acceptable salt thereof), and a pharmaceutically acceptable carrier to a subject in need of such treatment. A therapeutically effective amount of each of the component of the combination of the present invention may be administered simultaneously or sequentially and in any order. The method as herein defined, comprising administering: (i) an amount of 120 μg to about 250 μg, of about 140 μg to about 200 μg of tropifexor, and (ii) an amount of about 150 mg of cenicriviroc. Preferably, the method as herein defined, comprising administering: (i) an amount of about 140 μg of tropifexor, and (ii) an amount of about 150 mg of cenicriviroc.

A yet further aspect of the present invention is a medicine for a fibrotic disease or disorder, e.g. a liver disease or disorder, e.g. chronic liver disease or disorder, selected from the group consisting of NAFLD, NASH, liver fibrosis, cirrhosis and PBC, e.g. NASH, liver fibrosis or PBC containing active ingredients tropifexor, and (ii) cenicriviroc (in free form or as a pharmaceutically acceptable salt, solvate, prodrug, and/or ester thereof, e.g. in free form or as a pharmaceutically acceptable salt thereof) in combination.

In some preferred embodiments, the new dosing regimens of the combinations of the active ingredients disclosed herein are:
a) tropifexor, is to be administered at a dose (e.g. daily dose) of about 120 μg to about 250 μg, about 140 μg to about 200 μg,
b) cenicriviroc (in free form or as a pharmaceutically acceptable salt, solvate, prodrug, and/or ester thereof, e.g. in free form or as a pharmaceutically acceptable salt thereof), e.g. cenicriviroc mesylate is to be administered at a dose (e.g. daily dose) of about 150 mg, e.g. at a dose of 150 mg.

Such new combinations of the active ingredients as disclosed herein, are effective and well tolerated regimens for treating or preventing liver diseases and disorders mediated by farnesoid X receptors (FXR) in humans, e.g. NAFLD or NASH. The combinations described herein have additive effects to address the multifactorial etiology of NASH and provide an effective therapy in a large proportion of patients with NASH.

Various (enumerated) embodiments of the invention are described herein. It will be recognized that features specified in each embodiment may be combined with other specified features to provide further embodiments of the present invention.

### DETAILED DESCRIPTION OF THE INVENTION

### Definitions

For purposes of interpreting this specification, the following definitions will apply and whenever appropriate, terms used in the singular will also include the plural and vice versa.

As used herein, the term “about” in relation to a numerical value x means +/−10%, unless the context dictates otherwise.

As used herein, the term “FXR agonist” refers to an agent that directly binds to and upregulates the activity of FXR.

As used herein, the terms “salt” or “salts” refers to an acid addition or base addition salt of a compound of the invention. “Salts” include in particular “pharmaceutical acceptable salts”.

As used herein, the term “pharmaceutically acceptable” means a nontoxic material that does not interfere with the effectiveness of the biological activity of the active ingredient(s).

As used herein the term “prodrug” refers to compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “patient” or “subject” refer to a human.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

For example, “treating” NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation.

“Treating” or “treatment” of NAFLD or NASH in a human includes one or more of:
a) Preventing or reducing the risk of developing NAFLD or NASH, i.e., causing clinical symptoms of NAFLD or NASH not to develop in a subject who may be predisposed to NAFLD or NASH
b) Inhibiting NAFLD or NASH, i.e., arresting or reducing the development of NALFD or NASH or its clinical symptoms; and
c) Relieving NAFLD or NASH, i.e., causing regression, reversal, or amelioration of the NAFLD or NASH or reducing number, frequency, duration or severity of its clinical symptoms.

As used herein, the term “therapeutically effective amount” refers to an amount of the compound of the invention, e.g. tropifexor (as herein defined, e.g. in free form or as a stereoisomer, an enantiomer, a pharmaceutically acceptable salt, solvate, prodrug, ester thereof and/or an amino acid conjugate thereof), or cenicriviroc (in free form or as a pharmaceutically acceptable salt, solvate, prodrug, and/or ester thereof, e.g. in free form or as a pharmaceutically acceptable salt thereof), which is sufficient to achieve the stated effect. Accordingly, a therapeutically effective amount used for the treatment or prevention of a liver disease or disorder as hereinabove defined is an amount sufficient for the treatment or prevention of such a disease or disorder.

By “therapeutic regimen” is meant the pattern of treatment of an illness, e.g., the pattern of dosing used during the treatment of the disease or disorder.

As used herein, a subject is “in need of” a treatment if such subject would benefit biologically, medically or in quality of life from such treatment.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where a FXR agonist tropifexor of the present invention and cenicriviroc or a pharmaceutically acceptable salt or solvate thereof, also referred to as or “co-agent”) may be administered independently at the same time or separately within time intervals, especially where these time intervals allow that the combination partners show a cooperative, e.g. synergistic effect.

The terms “co-administration” or “combined administration” or the like as utilized herein are meant to encompass administration of the additional therapeutic agent to a single subject in need thereof (e.g. a patient), and the additional therapeutic agent are intended to include treatment regimens in which the FXR agonist tropifexor and cenicriviroc are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients, i.e. i) a non-bile acid derived FXR agonist tropifexor (in free form or e.g. as a pharmaceutically acceptable salt or an amino acid conjugate thereof) and ii) cenicriviroc (as herein defined, e.g. cenicriviroc mesylate), are both administered to a patient simultaneously in the form of a single entity or dosage.

The term “free combination” means that the active ingredients as hereindefined are both administered to a patient as separate entities either simultaneously, concurrently or sequentially with no specific time limits, and in any order, wherein such administration provides therapeutically effective levels of the two compounds in the body of the patient.

By “simultaneous administration”, it is meant that the FXR agonist tropifexor and cenicriviroc (as herein defined, e.g. cenicriviroc mesylate), are administered on the same day. The two active ingredients can be administered at the same time (for fixed or free combinations) or one at a time (for free combinations).

According to the invention, “sequential administration”, may mean that during a period of two or more days of continuous co-administration only one of tropifexor and cenicriviroc (as herein defined, e.g. cenicriviroc mesylate), is administered on any given day.

By “overlapping administration”, it is meant that during a period of two or more days of continuous co-administration, there is at least one day of simultaneous administration and at least one day when only one of tropifexor and cenicriviroc (as herein defined, e.g. cenicriviroc mesylate), is administered.

By “interval administration”, it is meant a period of co-administration with at least one void day, i.e. with at least one day where neither tropifexor nor cenicriviroc (as herein defined, e.g. cenicriviroc mesylate, is administered.

By “continuous administration”, it is meant a period of co-administration without any void day. The continuous administration may be simultaneous, sequential, or overlapping, as described above.

As used herein, the term “qd” means a once daily administration.

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Diseases

As hereinabove defined, the fibrotic or cirrhotic disease or disorder can be a liver disease or disorder, e.g. as defined below herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis.

The liver diseases or disorders can also refer to liver transplantation.

In one embodiment of the invention, the pharmaceutical combination (as herein defined) is for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a pharmaceutical combination of the invention for the improvement of liver fibrosis without worsening of steatohepatitis.

In another embodiment of the invention, there is provided a pharmaceutical combination of the invention for obtaining a complete resolution of steatohepatitis without worsening, e.g. improving, of liver fibrosis.

In another embodiment of the invention, there is provided a pharmaceutical combination of the invention for preventing or treating steatohepatitis and liver fibrosis.

In yet another embodiment of the invention, there is provided a pharmaceutical combination of the invention for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a pharmaceutical combination of the invention for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a pharmaceutical combination for treating or preventing, stage 3 fibrosis to stage 1 fibrosis, e.g. stage 3 and/or stage 2 and/or stage 1 fibrosis.

### Patients

According to the invention, the patients receiving the combination of the invention can be affected or at risk of a fibrotic disease or disorder, e.g. a liver disease or disorder, e.g. as hereinabove defined.

In some embodiments of the invention, the patient is obese or overweight

In other embodiments of the invention, the patient may be a diabetic patient, e.g. may have type 2 diabetes. The patient may have high blood pressure and/or high blood cholesterol level.

### Dosing Regimens

Depending on the patient general condition, the targeted disease or disorder and the stage of such disease or disorder, the dosing regimen, i.e. administered doses and/or frequency of each component of the pharmaceutical combination may vary.

The frequency of dosing of tropifexor and cenicriviroc, e.g. as a fixed dose combination, may be once per day, twice per day, three times per day, four times per day, five times per day, six times per day, or every two days, every three days or once per week, e.g. once a day.

According to the invention, tropifexor and cenicriviroc, both as herein defined, may not be administered following the same regimen, i.e. may not be administered at the same frequency and/or duration and/or dosage, e.g. at the same frequency and/or dosage. This can be the case e.g. for free combinations.

In one embodiment, e.g. in case of simultaneous administration, tropifexor (as hereinabove defined) is administered one to four times per day, and cenicriviroc (as hereinabove defined) is administered from one to four times per day.

In one embodiment of the invention, the co-administration is carried out for at least one week, at least one month, at least 6 weeks, at least three months, at least 6 months, at least one year. For example, the pharmaceutical combination of the invention is administered lifelong to the patient. The frequency of administration, and/or the doses of the tropifexor and of cenicriviroc, may vary during the whole period of administration.

In case of a sequential co-administration, tropifexor (as hereinabove defined) may be administered prior to cenicriviroc (as hereinabove defined), or reciprocally. The time interval between administration of tropifexor and of cenicriviroc may vary from a few minutes to a few days, e.g. a few minutes, e.g. a few hours, e.g. 1 day to 1 week.

The dosing frequency will depend on, inter alia, the phase of the treatment regimen.

According to the invention, tropifexor (as hereinabove defined), is administered at a dose of about 120 μg to about 250 μg, e.g. about 140 μg to about 200 μg. Such doses may be for oral administration. Such doses may be for daily administration, or twice daily administration or every two days administration, e.g. for daily oral administration, twice daily oral administration or every two days oral administration. Tropifexor (as hereinabove defined), is administered at a dose of 140 μg.

In some aspects, tropifexor (as herein above defined) that is administered with cenicriviroc (in free form or as a pharmaceutically acceptable salt, solvate, prodrug and/or ester thereof, e.g. cenicriviroc mesylate), is administered at a dose of about 120 μg, about 140 μg, or about 200 μg. Such doses may be for daily or twice daily, e.g. for daily administration. Such doses are particularly adapted for oral administration of tropifexor.

In some embodiments, tropifexor, as herein defined, is administered at a dose of about 120 μg delivered orally, about 140 μg delivered orally or about 200 μg delivered orally. Such doses may be for oral administration.

In some embodiments, tropifexor as herein defined, is to be administered at a daily dose of about 120 μg.

In some embodiments, tropifexor as herein defined, is to be administered at a daily dose of about 140 μg.

In some embodiments, tropifexor as herein defined, is to be administered at a daily dose of about 200 μg.

According to the invention, cenicriviroc (as hereinabove defined, e.g. cenicriviroc mesylate) is administered at a dose of about 50 mg, e.g. about 60 mg, e.g. about 80 mg, e.g. about 100 mg, e.g. about 120 mg, e.g. about 140 mg, e.g. about 150 mg, e.g. about 180 mg, e.g. about 200 mg, e.g. about 220 mg, e.g. about 250 mg. Such doses may be for oral administration cenicriviroc (as hereinabove defined, e.g. cenicriviroc mesylate). Such doses may be for daily administration of cenicriviroc (as hereinabove defined, e.g. cenicriviroc mesylate), twice daily administration or every two days administration, e.g. for daily oral administration.

In some aspects, cenicriviroc (as herein above defined, e.g. cenicriviroc mesylate) is administered at a dose in a range of about 30 mg to about 250 mg, e.g. about 50 mg to about 250 mg, e.g. about 100 mg to about 250 mg, e.g. about 10 mg to about 200 mg; e.g. about 100 mg to about 200 mg; e.g. about 30 mg to about 200 mg, e.g. about 50 mg to about 200 mg. Such doses may be for oral administration cenicriviroc (as hereinabove defined, e.g. cenicriviroc mesylate). Such doses may be for daily administration of cenicriviroc (as hereinabove defined, e.g. cenicriviroc mesylate), twice daily administration or every two days administration, e.g. for daily oral administration.

In some embodiments, cenicriviroc (as hereinabove defined, e.g. cenicriviroc mesylate), is administered at a dose of about 50 mg delivered orally, about 60 mg delivered orally, about 80 mg delivered orally, about 100 mg delivered orally, about 120 mg delivered orally, about 140 mg delivered orally, about 150 mg delivered orally, about 180 mg delivered orally, about 200 mg delivered orally, about 220 mg delivered orally, about 250 mg delivered orally. Such doses may be particularly adapted for patients of weight between 50 and 120 kg, e.g. 70 and 100 kg.

In some embodiments, cenicriviroc (as hereinabove defined, e.g. cenicriviroc mesylate), is administered at a dose in a range of about 50 mg/day, e.g. about 60 mg/day, e.g. about 80 mg/day, e.g. about 100 mg/day, e.g. about 120 mg/day, e.g. about 140 mg/day, e.g. about 150 mg/day, e.g. about 180 mg/day, e.g. about 200 mg/day, e.g. about 220 mg/day, e.g. about 250 mg/day. Such regimens may be delivered orally. Such regimens may be particularly adapted for patients of weight between 50 and 120 kg, e.g. 70 and 100 kg.

In some embodiments of the invention, cenicriviroc (as hereinabove defined, e.g. cenicriviroc mesylate), is administered at a dose of about 50 mg twice daily, about 60 mg twice daily, about 80 mg twice daily, about 100 mg twice daily, about 140 mg twice daily, about 150 mg twice daily, about 180 mg twice daily, about 200 mg twice daily, about 220 mg twice daily, about 250 mg twice daily. Such regimens may be delivered orally.

In one embodiment of the invention, the pharmaceutical combination, e.g. fixed or free combination, comprises i) 120 μg to about 250 μg, e.g. about 140 μg to about 200 μg of tropifexor and ii) about 100 mg to about 250 mg of cenicriviroc (as hereinabove defined, e.g. cenicriviroc mesylate). For example, the pharmaceutical combination, e.g. fixed or free combination, comprises i) about 140 μg of tropifexor (as hereinabove defined) and ii) about 150 mg of cenicriviroc (as hereinabove defined, e.g. cenicriviroc mesylate). For example, the pharmaceutical combination, e.g. fixed or free combination, comprises i) about 200 μg of tropifexor and ii) about 150 mg of cenicriviroc (as hereinabove defined, e.g. cenicriviroc mesylate).

### Kits for the Treatment of Fibrotic Disease or Disorder, e.g. a Liver Disease or Disorder

Accordingly, there are provided pharmaceutical kits comprising: a) tropifexor (as hereinabove defined) and b) cenicriviroc (as hereinabove defined, e.g. cenicriviroc mesylate); and c) means for administering tropifexor (as herein defined) and cenicriviroc (as hereinabove defined, e.g. cenicriviroc mesylate), to a subject affected by a liver disease or disorder; and optionally d) instructions for use.

In one embodiment of the invention, there is provided a combination package comprising a) at least one individual dose of tropifexor as herein defined; and b) cenicriviroc (as hereinabove defined, e.g. cenicriviroc mesylate). The combination package may further comprise instructions for use.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### Example 1

A 1-month combination toxicity study was performed with cenicriviroc (CVC) (30 mg/kg/day) and tropifexor (TRX) (0.03 mg/kg or 1 mg/kg/day). Administration of CVC alone caused no microscopic findings indicative of toxicity and administration of CVC in combination with tropifexor had no impact on toxicity of TRX.

A total of 69 subjects have received TRX at doses ranging from 10 μg to 3000 μg in single or 10 μg to 100 μg in multiple daily doses. Dose-dependent increases in FGF19, a biomarker of FXR target engagement in the enterocyte, were noted in single and multiple dose studies. No safety concerns were identified in single dose studies up to 3000 μg TRX.

CVC was generally well-tolerated in Phase I studies evaluating single doses of CVC up to 900 mg and at multiple daily doses of up to 400 mg for 10 days.

### Study Design

This is a single site, randomized, parallel group, 3 arm double-blind, double-dummy study to evaluate the potential drug-drug interaction between TRX and CVC in healthy and healthy but overweight to obese subjects. Healthy and healthy but overweight to obese subjects with body mass index (BMI) up to 32, are included to give a better representation of the NASH patients that will be studied in future clinical studies. The subjects were randomized into 3 arms a) TRX monotherapy, b) CVC monotherapy, and c) TRX and CVC as a combination.

A total of 42 subjects were randomized into the 3 parallel arms receiving single daily doses of TRX, CVC, and TRX+CVC combination.

This study examined doses of 60 μg of tropifexor and 150 mg of CVC given with a standard breakfast.

Pharmacokinetics: Sample collections included time points up to 24 hours after the first (Day 1) and the last (Day 14) doses to establish the AUC0-24 h,Cmax AUC0-24 h,ss and Cmax,ss for TRX, CVC, and TRX+CVC plasma concentration-time profiles.

### Results

The combination therapy was well tolerated with a similar profile to that observed of the monotherapies. When dosed alone, Day 1 TRX observed peak drug concentration (Cₘₐₓ) and area under the concentration-time curve (AUC_{0-24 h}) values were 1.46 ng/mL and 17.9 ng/mL*hr, respectively. Co-administration of CVC decreased Day 1 Cₘₐₓ and AUC_{0-24 h} of TRX by 33% and 32%, respectively. On Day 14, steady-state C_{max,ss} and AUC_{0-24 h,ss} of TRX were 1.77 ng/mL and 25.2 ng/mL*hr, respectively, and both reduced by 35% when co-administered with CVC. When dosed alone, Day 1 CVC Cₘₐₓ and AUC_{0-24 h} were 450 ng/mL and 4,645 ng/mL*hr, respectively. CVC accumulated in plasma over the study duration, with Day 14 CVC C_{max,ss} and AUC_{0-24 h,ss} values of 778 ng/mL and 10,830 ng/mL*hr, respectively. Co-administration with TRX had no marked impact on Cₘₐₓ and AUC_{0-24 h} of CVC.

Based on the above safety results showing that co-administration of CVC reduces TRX exposure by 32-33%, it is proposed to use higher doses of TRX when co-administered with CVC; e.g. doses of 140 μg and of 200 μg of TRX when co-administered with 150 mg CVC.

### Example 2

TANDEM (NCT03517540) is an ongoing, 48-week, Phase 2b, multicenter, randomized, double-blind study assessing the safety, tolerability and efficacy of a combination of TRX and CVC in patients with NASH and liver fibrosis (Stage 2 or 3 [F2/F3] as per the NASH clinical research network [CRN] scoring system) with a planned recruitment target of 200 patients

The study starts with an 10-week screening period, after which eligible patients are randomized (1:1:1:1) to one of the following four treatment arms: (1) TRX 140 μg qd, (2) CVC 150 mg qd, (3) qd combination of TXR 140 μg+CVC 150 mg, or (4) qd combination of TXR 90 μg+CVC 150 mg. The patients are treated for 48 weeks with a further follow-up period of 4 weeks.

### Study Population

Key inclusion criteria: Male and female patients aged ≥18 years with weight ranging from 50-200 kg. Biopsy confirmed NASH with liver fibrosis F2 or F3 according to NASH CRN criteria

### Key Exclusion Criteria:

Previous exposure to investigational drugs in NASH;

Previous participation in a clinical trial and exposure to any investigational product being evaluated for the treatment of liver fibrosis or NASH in the 6 months before screening;

Use of medications prohibited by the protocol;

Current or history of significant alcohol consumption (males, >30 g/day; females, >20 g/day, on average) for a period of >3 consecutive months within 1 year prior to screening and/or a score on the modified alcohol use disorders identification test (AUDIT) questionnaire ≥8;

Uncontrolled diabetes defined as HbA1c ≥9% at screening;

History of treated or untreated malignancy of any organ except for basal cell carcinoma of the skin or treated cervical intraepithelial neoplasia, within the past 5 years;

History of autoimmune liver disease, inflammatory bowel disease, other forms of chronic liver disease, or liver transplantation;

Previous hepatic decompensation or severe liver impairment;

Calculated estimated glomerular filtration rate (by Modification of diet in renal disease formula) <60 mL/min;

Patients who are not candidates for liver biopsy;

History or current diagnosis of cirrhosis.

1-10. (canceled)

11. A method of preventing, delaying or treating a chronic liver disease or disorder comprising simultaneously or sequentially administering to a patient in need thereof about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a stereoisomer, enantiomer, pharmaceutically acceptable salt, prodrug, ester thereof or amino acid conjugate thereof; in combination with about 150 mg of cenicriviroc or a pharmaceutically acceptable salt, solvate, prodrug or ester thereof;
wherein said chronic liver disease or disorder is selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), nonalcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, diabetes, diabetic nephropathy, colitis, newborn jaundice, kernicterus, veno-occlusive disease, portal hypertension, metabolic syndrome, hypercholesterolemia or progressive fibrosis of the liver.

12. The method of claim 11 for treating non-alcoholic fatty liver disease (NAFLD), non-alcoholic steatohepatitis (NASH) or primary biliary cirrhosis (PBC) comprising simultaneously or sequentially administering to a patient in need thereof about 140 μg or about 200 μg of 2-[(1R,3r,55)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; in combination with about 150 mg of cenicriviroc or a pharmaceutically acceptable salt thereof.

13. The method of claim 12 for treating non-alcoholic fatty liver disease (NAFLD).

14. The method of claim 12 for treating non-alcoholic steatohepatitis (NASH).

15. The method of claim 12 for treating primary biliary cirrhosis (PBC).

16. The method of claim 12, comprising simultaneously or sequentially administering to a patient in need thereof about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; in combination with about 150 mg of cenicriviroc or a pharmaceutically acceptable salt thereof.

17. The method of claim 16, comprising simultaneously or sequentially administering to a patient in need thereof about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; in combination with about 150 mg of cenicriviroc mesylate.

18. The method of claim 12, comprising simultaneously or sequentially administering to a patient in need thereof about 140 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; in combination with about 150 mg of cenicriviroc or a pharmaceutically acceptable salt thereof.

19. The method of claim 18, comprising simultaneously or sequentially administering to a patient in need thereof about 140 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; in combination with about 150 mg of cenicriviroc mesylate.

20. The method of claim 12, comprising simultaneously or sequentially administering to a patient in need thereof about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; in combination with about 150 mg of cenicriviroc or a pharmaceutically acceptable salt thereof.

21. The method of claim 20, comprising simultaneously or sequentially administering to a patient in need thereof about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; in combination with about 150 mg of cenicriviroc mesylate.

22. A pharmaceutical combination comprising about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a stereoisomer, enantiomer, pharmaceutically acceptable salt, prodrug, ester thereof or amino acid conjugate thereof; and about 150 mg of cenicriviroc or a pharmaceutically acceptable salt, solvate, prodrug or ester thereof.

23. The pharmaceutical combination of claim 22, comprising about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; and about 150 mg of cenicriviroc or a pharmaceutically acceptable salt thereof.

24. The pharmaceutical combination of claim 22, comprising about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; and about 150 mg of cenicriviroc or a pharmaceutically acceptable salt thereof.

25. The pharmaceutical combination of claim 24, comprising about 140 μg or about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; and about 150 mg of cenicriviroc mesylate.

26. The pharmaceutical combination of claim 22, comprising about 140 μg of 2-[(1R ,3r, 5S)-3-({5-cyclopropyl-3-[2-(trifluoromethoxy)phenyl]- 1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; and about 150 mg of cenicriviroc or a pharmaceutically acceptable salt thereof.

27. The pharmaceutical combination of claim 26, comprising about 140 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; and about 150 mg of cenicriviroc mesylate.

28. The pharmaceutical combination of claim 22, comprising about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid or a pharmaceutically acceptable salt thereof; and about 150 mg of cenicriviroc or a pharmaceutically acceptable salt thereof.

29. The pharmaceutical combination of claim 28, comprising about 200 μg of 2-[(1R,3r,5S)-3-({5-cyclopropyl-3-[2-(trifluoromethoxy)phenyl]-1,2-oxazol-4-yl}methoxy)-8-azabicyclo [3.2.1]octan-8-yl]-4-fluoro-1,3-benzothiazole-6-carboxylic acid in free form; and about 150 mg of cenicriviroc mesylate.

30. The pharmaceutical combination of claim 22, wherein said combination is a fixed combination.

31. The pharmaceutical combination of claim 22, wherein said combination is a free combination.
